(19) United States
(12) Patent Application Publication
(10) Pub. No.: US 2024/0386570 A1
(43) Pub. Date: Nov. 21, 2024

(54) 3D QUANTITATIVE JOINT MUSCLE EVALUATION VIA AUTOMATED JOINT MUSCLE SEGMENTATION WITH ARTIFICIAL INTELLIGENCE

(71) Applicant: The Cleveland Clinic Foundation, Cleveland, OH (US)

(72) Inventors: Mingrui Yang, Cleveland, OH (US); Bong-Jae Jun, Cleveland, OH (US); Joshua Polster, Cleveland, OH (US); Carl Winalski, Cleveland, OH (US); Kathleen Derwin, Cleveland, OH (US); Eric T. Ricchetti, Cleveland, OH (US); Xiaojuan Li, Cleveland, OH (US); Nicolas Piuzzi, Cleveland, OH (US)

(21) Appl. No.: 18/667,194

(22) Filed: May 17, 2024

Related U.S. Application Data
(60) Provisional application No. 63/467,777, filed on May 19, 2023.

(51) Int. Cl. G06T 7/10; A61B 5/00; A61B 5/107; G06T 7/00; G06T 7/62; G06V 10/26; G06V 10/28; G06V 10/764; G16H 50/50

(52) U.S. Cl. CPC G06T 7/10; A61B 5/1073; A61B 5/4519; A61B 5/4576; A61B 5/458; A61B 5/4585; A61B 5/4872; G06T 7/0012; G06T 7/62; G06V 10/26; G06V 10/28; G06V 10/764; G16H 50/50; G06T 2207/10081; G06T 2207/10088; G06T 2207/20004; G06T 2207/20081; G06T 2207/30004; G06V 2201/033

(57) ABSTRACT

A muscle segmentation method and system utilizes a trained machine learning system to identify and segment skeletal muscle from input magnetic resonance images of the shoulder. Properties of the muscle can be determined from the segmentation, and clinical treatment decisions can be more accurately made based on the determined properties.

### CROSS-REFERENCE TO RELATED APPLICATIONS

This application claims priority to U.S. Provisional Patent Application No. 63/467,777 filed May 19, 2023 and entitled “3D QUANTITATIVE SHOULDER MUSCLE EVALUATION VIA AUTOMATED SHOULDER MUSCLE SEGMENTATION WITH ARTIFICIAL INTELLIGENCE”, the entirety of which is incorporated herein by reference.

### STATEMENT OF GOVERNMENT-SPONSORED RESEARCH

This invention was made with government support under AR075286 awarded by the National Institutes of Health. The government has certain rights in the invention.

### BACKGROUND

Muscle characteristics such as muscle quality and quantity play significant roles in musculoskeletal disorder diagnosis and treatment prognosis. Muscle volume and fat fraction are two characteristics clinicians use to define muscle quality and quantity. Separately, studies suggest that rotator cuff repair is not satisfactory for 20%-30% of patients because of muscle quality and quantity before surgery. For instance, muscle atrophy and fatty infiltration are significantly correlated with worse clinical outcomes after rotator cuff repair. Therefore, it can be important to identify muscle quality and quantity prior to any surgical decisions because muscle pathology is potentially modifiable—for example, with exercise training or pharmacological interventions—in a manner that can improve surgical outcomes. Similarly, muscle volume and fat fraction can impact surgical decision-making, including the type of surgical repair chosen or whether a patient is a good candidate for surgery overall.

One approach to determining volume of individual muscles involves muscle segmentation. Fat fraction can be calculated by using the signal arising from fat protons divided by the sum of the signals from fat and water protons. Quantifying muscle volume and fat fraction, along with other characteristics of muscle tissue, can be labor intensive and error prone. For instance, in some applications, an magnetic resonance (MR) image is first taken of the patient and analyzed by a group of clinicians to determine muscle volume and fat fraction. But this analysis of the MR image can include manually tracing boundaries, and collaborating with other clinicians to determine muscle boundaries.

Moreover, because muscle boundaries (including those in an MR image), are not always clear, determining muscle boundaries is difficult and can often require a group of clinicians for analysis. The group of clinicians typically need to agree on muscle boundaries before determining muscle volume and fat fraction. This group decision can be tiresome and difficult because clinicians do not always agree with the determined muscle boundaries. Even with a group of clinicians collaborating on muscle boundaries, this process can still be error prone and cause inaccurate muscle segmentation, which causes inaccurate estimates of muscle volume and fat fraction. Accordingly, manually segmentation of muscle tissue is not ideal and the analysis that can come therefrom is not always possible or accurate.

### BRIEF SUMMARY

According to one example of the present disclosure a method comprises: obtaining an image of skeletal muscles of a patient; inputting the image into a machine learning system trained to segment the muscles in the image; identifying a property of at least one of the skeletal muscles based on an identification of the segmented muscles; and determining a treatment procedure for the patient based on the identified property.

In various embodiments of the above example, the method further comprises: generating a visualization of the segmentation muscles as an output of the trained machine learning system, or based on the trained machine learning system; the image is a magnetic resonance image; the image is a computed tomography image; the image is in the sagittal plane; the identified property is a three-dimensional muscle volume; the muscle volume is determined based on a number of pixels or voxels within the segmented muscle; the identified property is a muscle quality; the muscle quality is a fat fraction of the muscle; the machine learning system is a multi-class classifier and is trained to segment at least three muscles of a shoulder of the patient; the method further comprises pre-processing the obtained image prior to inputting the image into the trained machine learning system; the pre-processing comprises performing a contrast limited adaptive histogram equalization (CLAHE); the skeletal muscles include a shoulder muscle; the skeletal muscles include a knee or thigh muscle; the skeletal muscles include an elbow muscle; the treatment procedure comprises determining a likelihood of success of a plurality of different treatment procedures and identifying the treatment procedure having the greatest likelihood of success; the image of skeletal muscles is a two-dimensional image from a computed tomography or magnetic resonance three-dimensional volume; the method comprises inputting a plurality of images into the machine learning system, the plurality of images being of a three-dimensional volume of the skeletal muscles, wherein the machine learning system is trained to segment the muscles in the three-dimensional volume; and/or the method comprises inputting a plurality of images into the machine learning system, the plurality of images being of a three-dimensional volume of the skeletal muscles, wherein the machine learning system is trained to output a three-dimensional volume in which the plurality of skeletal muscles are segmented.

According to another example of the present disclosure, a method comprises: obtaining a plurality of two-dimensional images of skeletal muscles of a patient, the plurality of two-dimensional images being of a three-dimensional volume obtained via computed tomography (CT) or magnetic resonance imaging (MRI); inputting the plurality of images into a machine learning system trained to segment the muscles in the image and to output a three-dimensional volume of segmented skeletal muscles; determining a fat fraction of at least one of the skeletal muscles based on an identification of the segmented muscles within the three-dimensional volume; determining a likelihood of success of a plurality of different treatment procedures based on the determined fat fraction; and identifying the treatment procedure having the greatest likelihood of success as a preferred treatment procedure for the patient.

### BRIEF DESCRIPTION OF THE SEVERAL VIEWS OF THE DRAWING

FIG. 1 illustrates an example muscle segmentation method of the present disclosure.

FIG. 2 illustrates example pre-processing of the present disclosure.

FIG. 3A illustrates an example muscle segmentation of a magnetic resonance image according to the present disclosure.

FIG. 3B illustrates an example muscle segmentation of a computed tomography image according to the present disclosure.

FIG. 4 illustrates an example training procedure for a machine learning system of the present disclosure.

FIG. 5 illustrates an example computing device for a machine learning system of the present disclosure.

### DETAILED DESCRIPTION OF THE DRAWING

Considering the above, the present disclosure relates to systems and methods for automated muscle segmentation. More particularly, the present disclosure relates to a system and method of deep learning based automated tool for three dimensional (3D) muscle volume segmentation. While the present disclosure is generally made herewith with respect to magnetic resonance (MR) images, it should be understood that the disclosure is also applicable to images from other modalities such as computed tomography (CT). Further, while the present disclosure is generally made herewith with respect to the shoulder, it should be understood that the disclosure is also applicable to other joints, such as the knee/thigh, elbow, or the like.

Using the methods and devices described below, muscle segmentation accuracy can be improved, leading to more accurate muscle volume quantification and subsequent fat fraction analysis. These methods and devices also substantially reduce the time required for 3D muscle volume and quality analysis. Ultimately, these methods and devices can provide a more accurate information to allow for a better prediction of clinical outcomes for patients and a valuable tool for clinicians.

Briefly, a musculoskeletal image of a patient (such as an MR image) is first obtained and optionally preprocessed. The preprocessed images are then input into a machine learning system trained to identify and segment muscles in the MR image. The machine learning system can then output images and/or data related to the segmentation of the muscles. The machine learning system can also output analysis related to the quality and quantity of the muscle, e.g., muscle volume and fat fraction. In particular, the fat fraction can be determined directly from the input image (either an MR or CT image). Moreover, the fat fraction or other characteristic may be determined within a two-dimensional slice/image or in a three-dimensional volume.

Depending on the embodiment, the machine learning system may be trained using musculoskeletal MR images of the same or similar joint. Generally, the systems and methods herein are directed toward evaluating muscles in the shoulder. Of course, the description is not limited to evaluating muscles in the shoulder, but can be used to determine muscle characteristics of any joint.

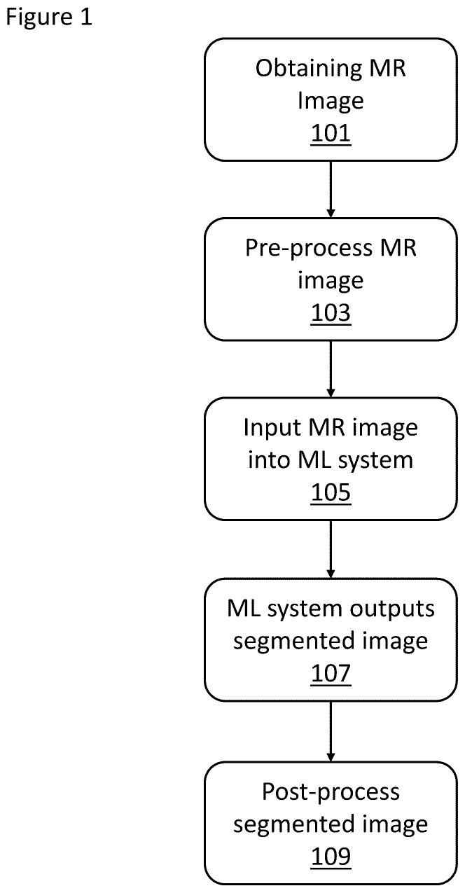

With reference to FIG. 1, the automated muscle segmentation 100 will be described in more detail. With particular reference to FIG. 1, the automated muscle segmentation method 100 first obtains an MR image 101. The MR image can be obtained using a MRI scanner or the like. The MR image can be in a sagittal plane, coronal plane, transverse plane, or any other planc or cross-sectional slice. The MR image can be T1-weighted, T2-weighted, or the like. The MR image may be a 2D image or a 3D volume (e.g., having 20-50 2D slices).

After the MR image is obtained 101, the MR image can then optionally be pre-processed 103. In particular it is noted that, MR images may benefit from more pre-processing than non-MR images. For example, because of the differences between CT images and MR images, CT images typically need the contrast adjusted. Pre-processing the MR image 103 can including modifying the brightness, contrast, sharpness, or the like area in a manner that increases visibility of specific regions or muscles of the MR image. Additionally, a region of interest may be identified with the MR image. For instance, the MR image can be cropped to only include the region of interest, or a region of interest may be fully extracted from the MR image. The region of interest may be a region having specific musculature or anatomy.

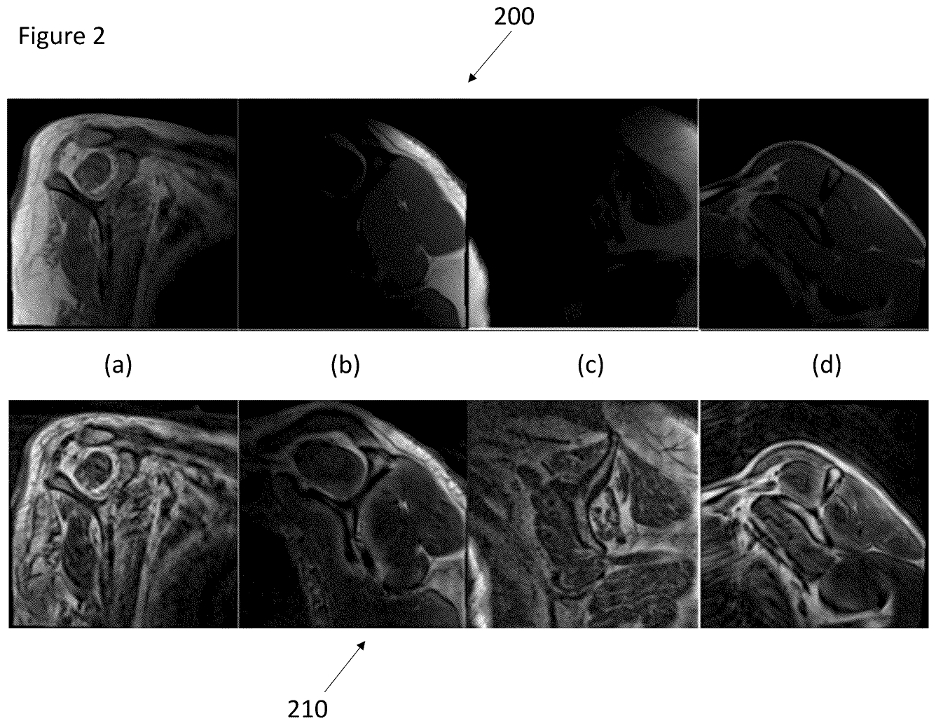

In one particular embodiment, MR images are pre-processed with a contrast limited adaptive histogram equalization (CLAHE) for better image contrast and homogeneity. CLAHE can also highlight muscles and highlight boundaries of the muscle by enhancing the contrast. The benefits of CLAHE are illustrated with respect to FIG. 2. As seen therein, originally obtained images 200 (a)-(d) are shown in the top panel and corresponding MR images preprocessed according to CLAHE 210 (a)-(d) are shown in the bottom panel. Comparing the original and processed MR images 200, 210, it can be seen that the CLAHE processed MR images 210 have enhanced details and are clearer than their respective obtained MR images 200.

Pre-processing can be performed by a user or automatically by an application, computer system, or machine learning system. In one embodiment, the pre-processing is included in the workflow of the execution of the machine learning system. For example, an unprocessed MR image may be input into the machine learning system, which is trained to first perform pre-processing techniques (such as increasing contrast) prior to segmentation. Such machine learning systems may be integrated or separate, such that a first machine learning system is trained to perform pre-processing techniques and to output a pre-processed image, which is then input to a second machine learning system trained to segment the pre-processed image.

After the image is determined to be suitable for the machine learning system analysis, the MR image is input into a machine learning (ML) system 105. The ML system is trained to segment the muscles found in the MR image. The ML system may perform single or multiple class segmentation (e.g., identifying and segmenting multiple muscles). In one embodiment, the ML system identifies and segments at least 3 muscles found in the shoulder. For instance, the supraspinatus, the infraspinatus, and the subscapularis. When the ML system is used with other joints/areas of the body, the ML system can identify at least the main muscles in that joint/area. In some embodiments, a plurality of individual two-dimensional images from a three-dimensional volume can be input into the ML system (e.g. sequentially or together).

In embodiments involving multiple classifications, the classifications may be part of one or more machine learning systems. In other words, the method may utilize one or more one class classifiers, or multi-class classifiers. In one example utilizing one class classifiers, the method utilizes three machine learning systems each independently trained to identify a different muscle. An input image may be input to the multiple machine learning systems in serial (e.g., where an output of a first classifier is input into a second classifier) or in parallel (e.g., where the same image is input to each of the classifiers).

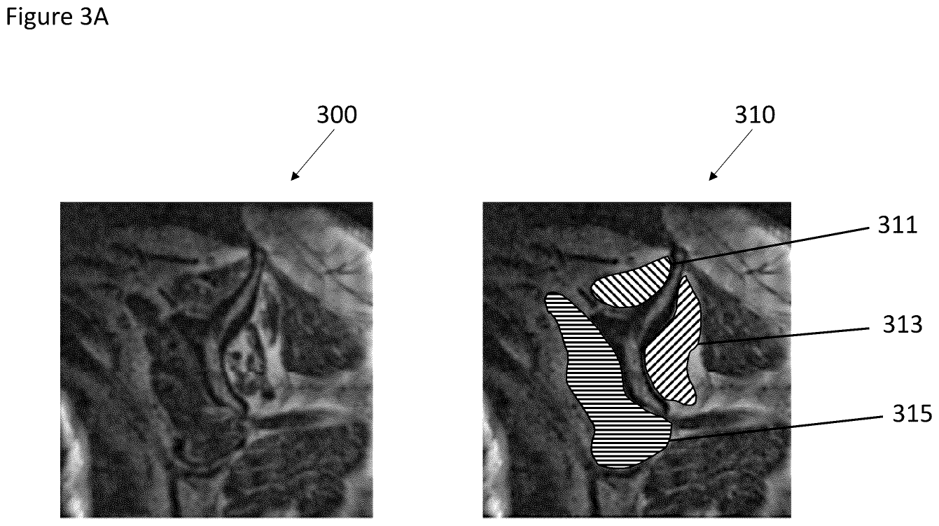

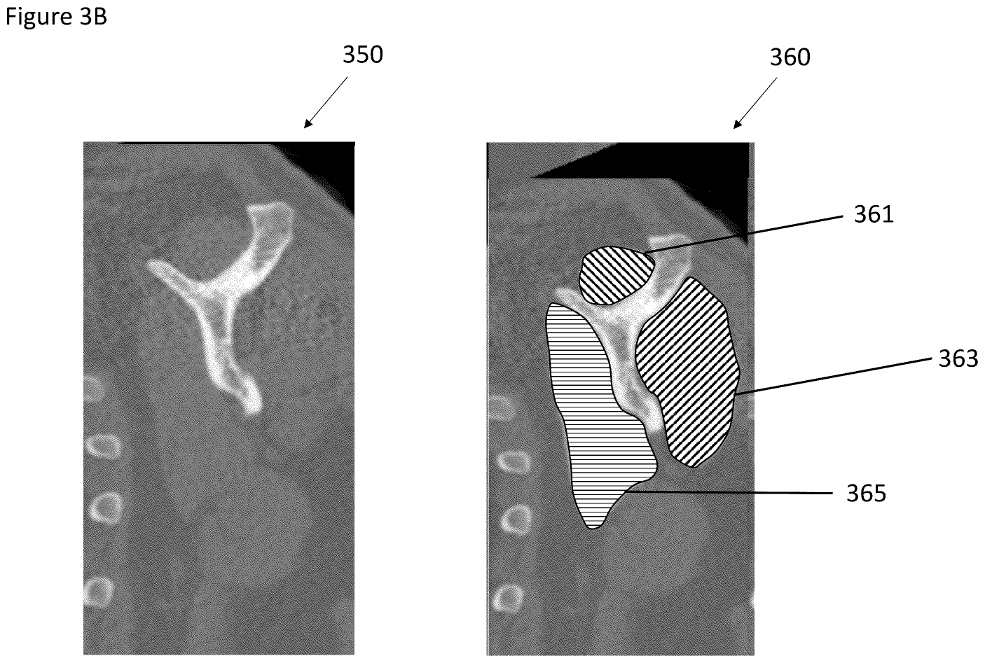

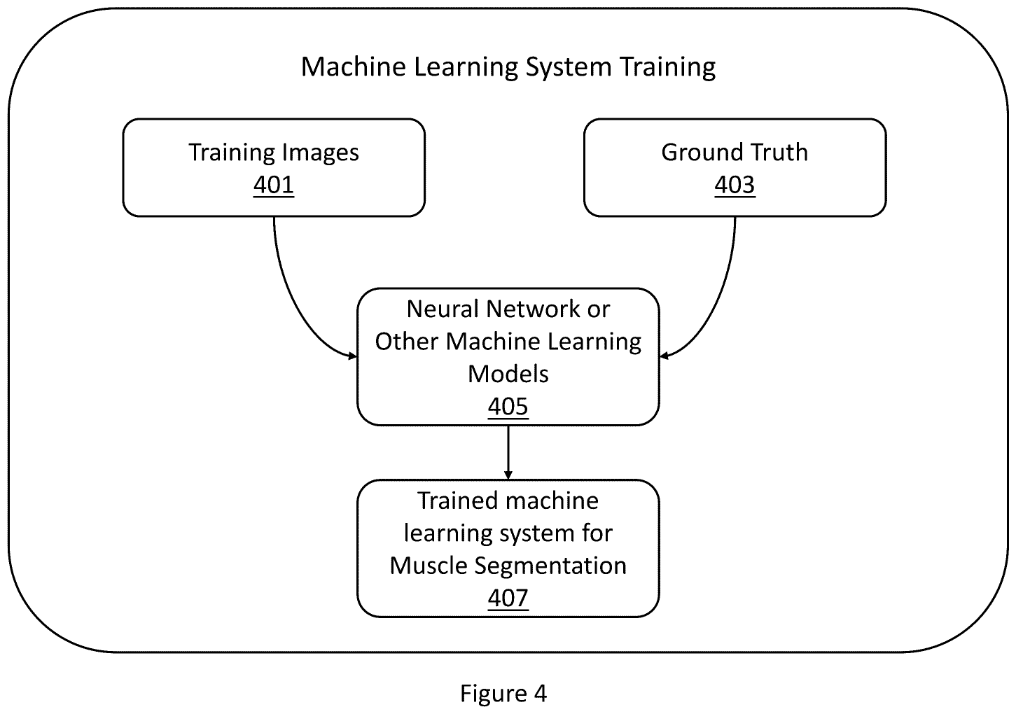

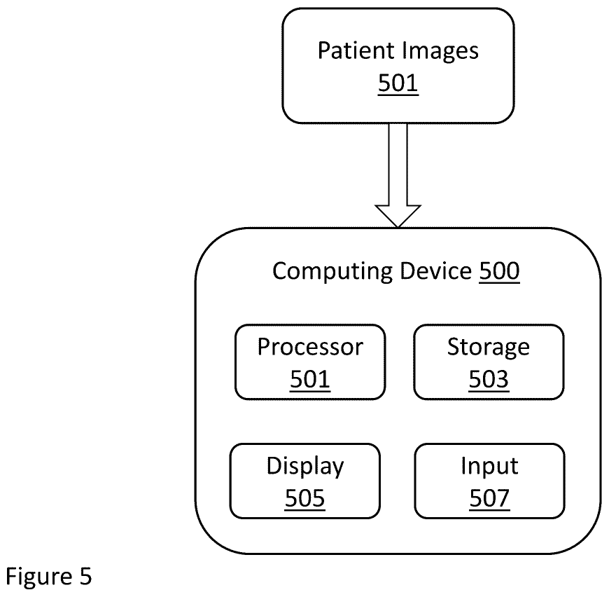

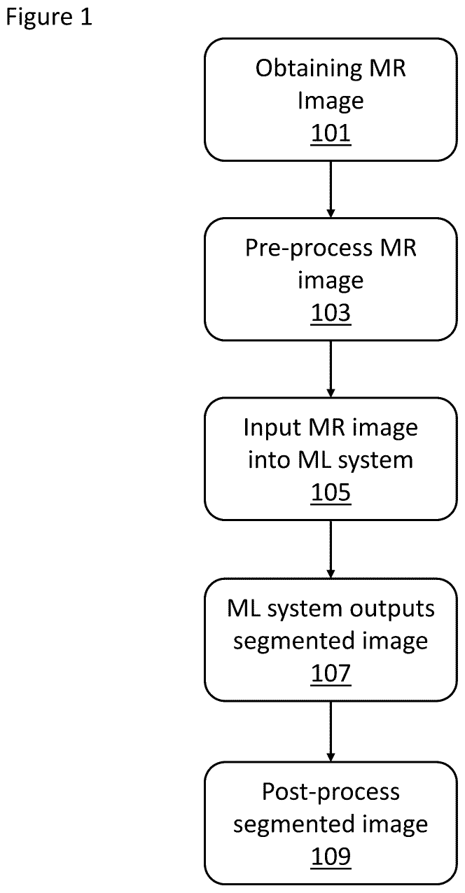

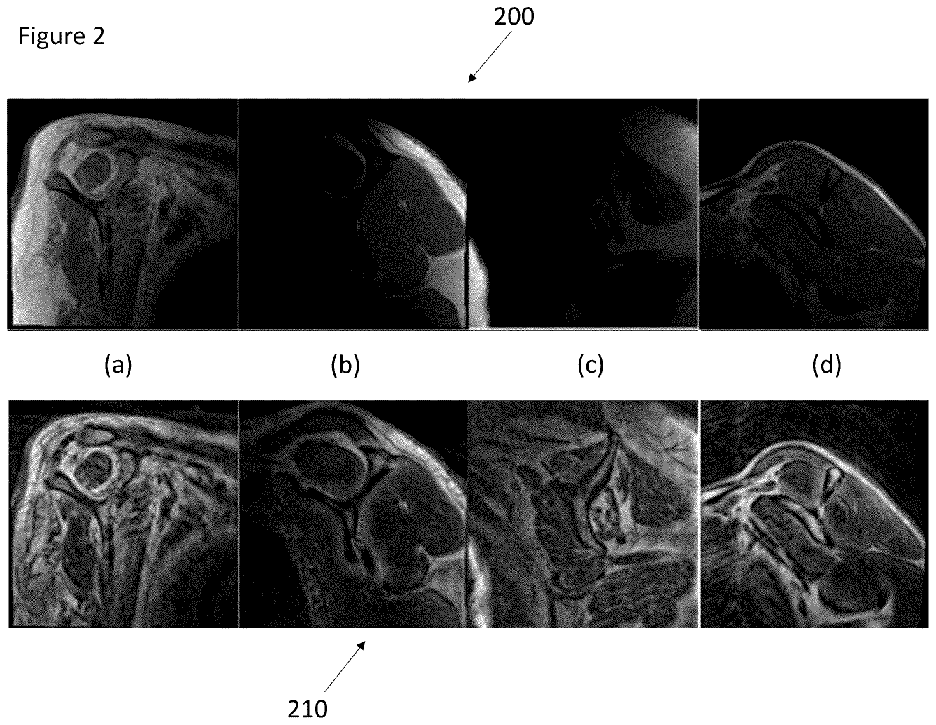

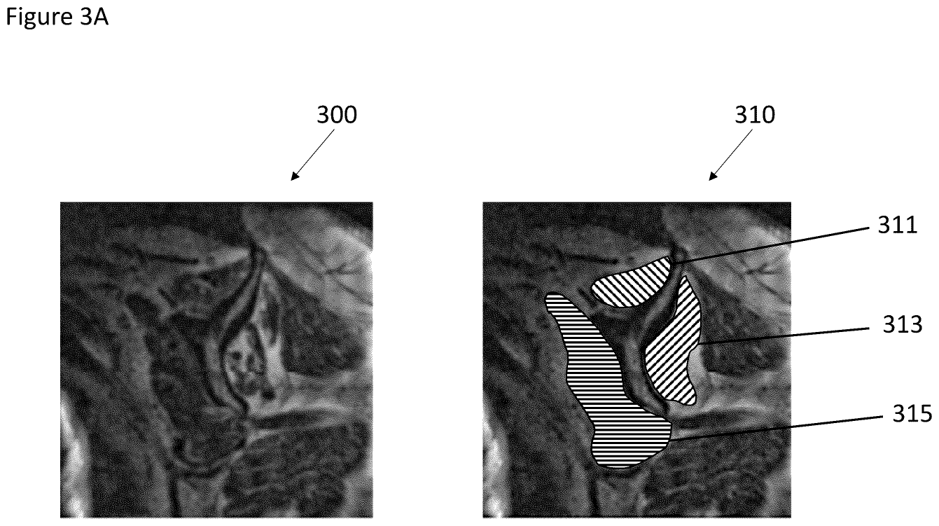

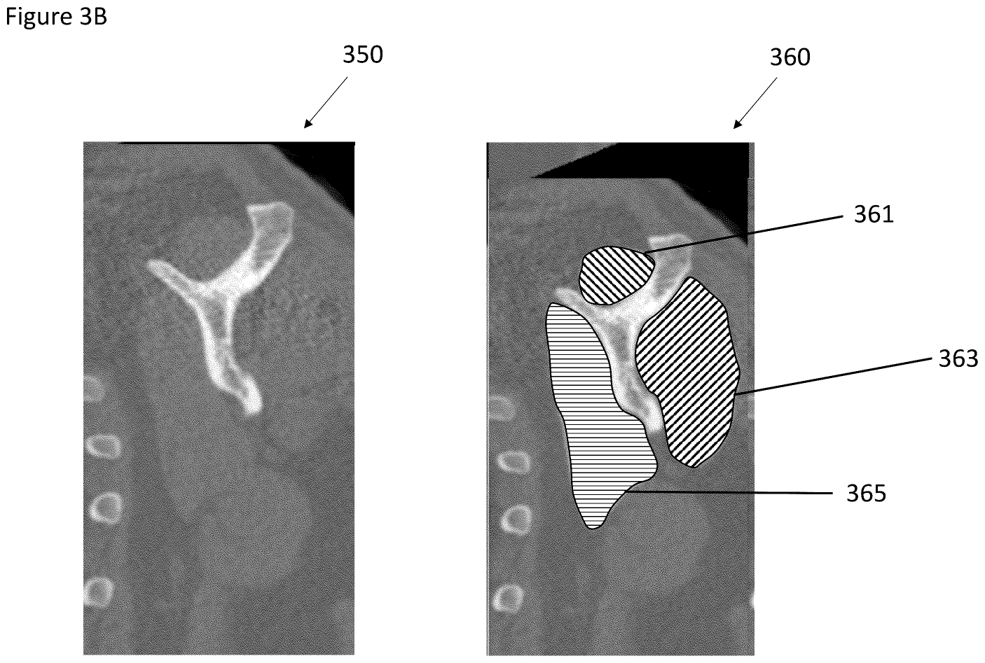

The ML system then outputs a segmented image 107. FIG. 3A illustrates a processed MR image 300 input into the ML system, and an output segmented image 310 indicating segmentation/outlining or highlighting of muscles. For instance, the segmented image 310 includes segmentation of three muscles; the supraspinatus 311, the infraspinatus/tres minor 313, and the subscapularis 315. Similarly, FIG. 3B illustrates a processed CT image 350 input into the ML system, and an output segmented image 360 indicating segmentation/outlining or highlighting of muscles including the supraspinatus 361, the infraspinatus/tres minor 363, and the subscapularis 365.

The muscle segmentation can be expressed using highlighting/shading of the muscles in different colors, or the like. The different muscles can also be labeled for case of understanding. The output images can include a key or legend indicating which color/shading corresponds to which muscle. In embodiments where a plurality of two-dimensional images are input (such as those form a three-dimensional volume), the output of the ML system can be a segmented three-dimensional volume or analysis of a three-dimensional volume. The three-dimensional output may be an aggregate of individual two-dimensional outputs, or may be a single three-dimensional output from the ML system.

Finally, the output segmented image 310 can be post-processed 109. Such post-processing may involve further image analysis and/or clinical analysis and may be performed automatically or by a clinician. For instance, the segmented output image 310 can be analyzed for quality and quantity of muscle to determine if the patient is suitable for surgery.

For example, fat fraction of a segmented muscle can be calculated using a Dixon sequence and/or modifications of a Dixon sequence including 2-point method, 3-point methods (Glover and IDEAL), echo-amplitude modulation, RF-phase alternation, direct phase encoding, orientation filtering, polynomial fitting, correction for chemical shift misregistration, and the like. Muscle volume can be calculated by determining the pixels (for a 2D image) or voxels (for 3D volume) that comprises the segmented muscle. That is, if the image pixel/voxel size (resolution) is known, volume is determined by identifying how many voxels comprise the segmented muscle and multiplying that number by the resolution. In view of this, the fat fraction can be determine in any manner directly from the input image (whether an MR, CT, or image from another modality)

Typically, calculating quality and quantity of muscle is not in the clinical routine because of the time and effort required. For instance, determining fat fraction based on the Dixon sequence, can be time consuming. However, by improving the accuracy of segmentation and reducing the time to segment, the present disclosure reduces this burden on the clinician. Similarly, by automating this process post-processing may further be automated thereby further reducing the burden on the clinician. As discussed above, this provides an opportunity for clinicians to predict if a patient is suitable for surgery.

Still further, it is envisioned that this analysis may be performed by comparison to normative databases. For example, any muscle properties identified via segmentation (e.g., volume, fat fraction, and the like) may be compared to those in a database of like properties from similar populations (e.g., age, gender, weight, height, and the like) and corresponding clinical outcomes or preferred clinical procedures. For instance, such a database may be used to identify that a reconstructive procedure has an above-average success rate for patients of a given age and gender and having given muscle volumes and fat fractions. This identification may be used to recommend the reconstructive procedure for that patient. However, if the database indicates a below-average success rate, a different rehabilitative procedure may instead be recommended for that patient.

In other words, depending on the embodiment, the analysis may include determining a likelihood of success of a given procedure and/or recommending a procedure having a highest likelihood of success or a likelihood of success above a predetermined threshold. This likelihood of success may be determined based on normative databases that relate the characteristic/fat fraction to success rates in patients having similar demographic information. Alternatively, the likelihood of success or desired procedure could be determined via machine learning systems trained to relate success rates to the characteristic and/or other demographic information about the patient. In these embodiments, the fat fraction or other characteristic of a given muscle and/or demographic information of a patient may be input to a machine learning system, and the machine learning system may output a likelihood of success of one or more treatment procedures, and/or may output a preferred treatment procedure. In some embodiments, a machine learning system may be trained to receive input MR or CT images, and to output a desired procedure or likelihood of success of a procedure directly without first outputting segmented muscles.

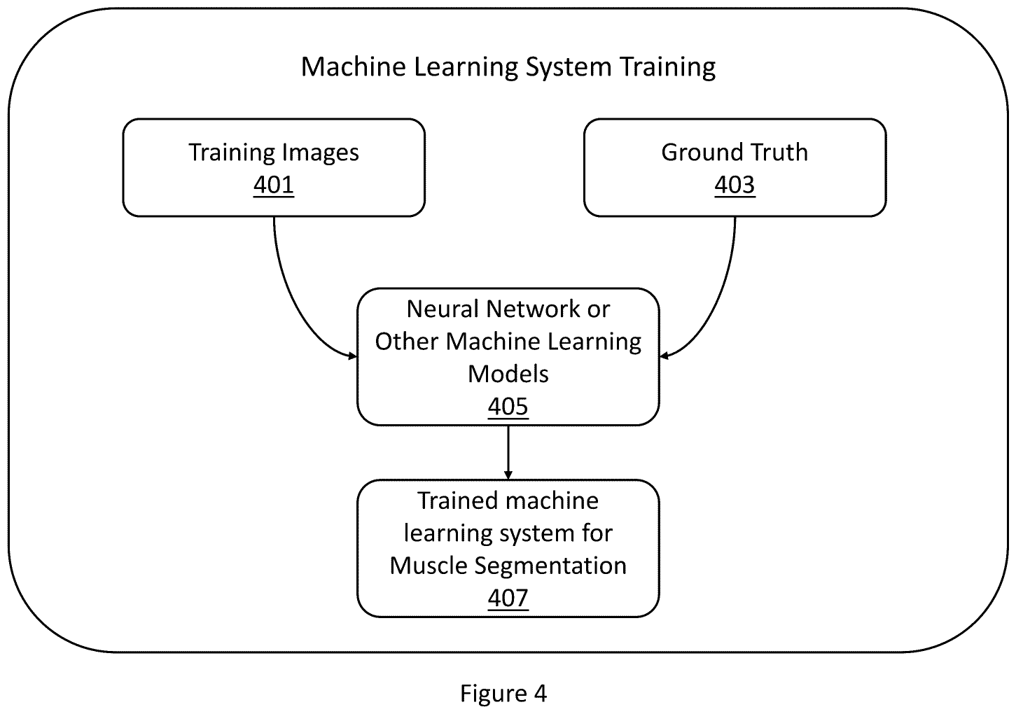

FIG. 4 illustrates training of any of the machine learning systems described herein. For example, a machine learning system can be trained to segment muscles 407 in images using supervised or unsupervised learning. In supervised learning, training images and the corresponding ground truth (e.g., segmentation) are input to the machine learning system. The training images are preferably of the same modality and plane that the trained system will receive. The ground truth may be identified by manual segmentation. The manual segmentation may be performed by a single clinician, or agreed to by a group of clinicians.

More particularly, the training images may be single slices of a 3D volume. In one example, the slices may be of the sagittal plane the shoulder. Images of the sagittal plane image are generally easily obtained, because the sagittal plane is typically already used and acquired in other clinical routines. In other embodiments the machine learning system can be trained on other images from other planes or cross-sectional slices, such as the coronal plane, transverse plane. The machine learning system can also be trained on images from multiple planes. Training a machine learning system with images from multiple planes can improve performance of the machine learning system and improve performance of the system with input images of multiple planes.

According to one particular example, the training images comprise thirty-four MR images of rotator cuff repair patients, including ten images with partial tears and fourteen images with full thickness tear, and ten images with no tear. The training images have various T1-weighted sequences on different MR scanners from different locations, with matrix sizes varying from 256 to 512, in-plane resolution varying from 0.23 mm to 0.86 mm, and slice thickness varying from 3 mm to 6 mm. The thirty-four MR images are then graded and segmented manually by a trained clinician or group of clinicians, for example, into three shoulder muscle groups (supraspinatus, combined infraspinatus/teres minor, and subscapularis).

The manually segmented training images are then randomly split into groups for training, validation, and testing. For instance, the thirty-four manually segmented images can be split into random groups of twenty-one images for training, seven for validation, and six for testing. These example groups provide 304, 103, and 74 slices, respectively. These groupings can also be random, or following traditional machine learning training/validation/testing standards, e.g., the 80/20 split, 75/20/5 split, or the like. As noted above, these images can also be pre-processed with CLAHE for better image contrast and homogeneity, and saved into 16-bit unsigned integer format. The MR images can further be augmented to help with training, validation, and testing, before being inputted in the learning model for training. For example, images can be randomly rotated, translated, zoomed, or the like.

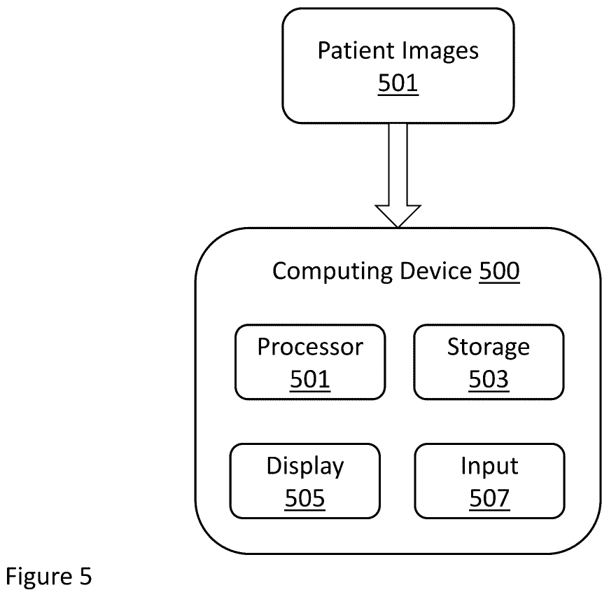

Turning to FIG. 5, the system described herein may be operated by a clinician using one or more computing devices 500. Each computing device may have a processor 501, storage 503 (e.g., of a non-transitory type), display 506 (e.g., a monitor, TV, screen, virtual/augmented reality headset, or the like), and input 507 (e.g., a mouse, keyboard, touch screen, voice controls, virtual/augmented reality controllers, or the like). The computing devices 500 can be a mobile device (such as a laptop, phone, or tablet) disposed in the vicinity of a patient for use by the clinician, tradition computer, server, or the like. Any of the one or more computing devices 500 may be located locally with (e.g., in a treatment room with the clinician and patient) or remotely. Further, any actions performed by the one or more computing devices 500 may be distributed in any manner. When more than one computing device 500 is utilized, those computing devices may be connected in any manner (e.g., wired or wireless, via local networks, the Internet, and the like)

In practice, the computing device 500 can comprise one or more of the machine learning systems described herein. A clinician can use the computing device 500 and provide/input a patient image 501. The computing device 500 can output an image illustrating the muscle segmentation and identifying muscles. This output image can be displayed on the display 505, or printed out for future review. The output image can be saved locally or off-premises (e.g., cloud based servers) in storage 503. The muscles in the output image can be highlighted or shaded to indicate different muscles. This output image can then be used by the clinician to determine muscle quality and quantity, as described above. This can be done manually by a clinician or by the computing device 500, or automatically by the computing device 500.

The computing device 500 can be connected to or communicate with an MR machine or the like. In one embodiment, the MR machine provides a recently taken MR image to the computing device 500, where the computing device uses that MR image as an input for the machine learning system. The machine learning system then identifies and segments at least one muscle from the image, and generates a visualization (e.g., displayed on the display 505, printed image, or stored image) for a clinician.

Similarly, the machine learning system(s) of the present disclosure may be embodied on computing devices remote from the user, and in communication with the user's computing device 500. In this manner, a single machine learning system may be accessible to a plurality of users (such as a plurality of clinicians within a single hospital or hospital system or even multiple hospitals or systems) without the need for training of multiple systems. In these instances, the machine learning system(s) may also be in communication with a plurality of MR or other imaging systems, or central repositories that store images acquired by those imaging systems. A command from a user can then cause the machine learning system to obtain appropriate input images from those imaging systems or repositories. In other embodiments, the user can directly send input images to the machine learning systems.

The above systems and methods are described with respect to 3D shoulder images, but can be applied to different areas and/or joints of a patient, such as a knee/thigh, elbow, or the like. Further, imaging modalities other than MR can be utilized, such as CT imaging. In those cases, the machine learning system is preferably trained with images from the desired area and with the desired modality.

According to the present disclosure, the time required for muscle segmentation is drastically reduced. Additionally, muscle segmentation accuracy is improved, along with the ease of performing muscle segmentation. With muscle segmentation accuracy and time improved, clinicians can better calculate muscle quality and quantity. The machine learning system of the present disclosure provides a valuable tool for quantifying 3D should muscle volume and subsequent fat fraction analysis to better predict clinical outcomes, e.g., surgery. Still further, these benefits are achievable independent of the training and expertise of the practicing clinician.

While various features are presented above, it should be understood that the features may be used singly or in any combination thereof. Further, it should be understood that variations and modifications may occur to those skilled in the art to which the claimed examples pertain.

What is claimed is:

1. A method comprising:
obtaining an image of skeletal muscles of a patient;
inputting the image into a machine learning system trained to segment the muscles in the image;
identifying a property of at least one of the skeletal muscles based on an identification of the segmented muscles; and
determining a treatment procedure for the patient based on the identified property.

2. The method of claim 1, further comprising:
generating a visualization of the segmentation muscles as an output of the trained machine learning system, or based on the trained machine learning system.

3. The method of claim 1, wherein the image is a magnetic resonance image.

4. The method of claim 1, wherein the image is a computed tomography image.

5. The method of claim 1, wherein the image is in the sagittal plane.

6. The method of claim 1, wherein the identified property is a three-dimensional muscle volume.

7. The method of claim 6, wherein the muscle volume is determined based on a number of pixels or voxels within the segmented muscle.

8. The method of claim 1, wherein the identified property is a muscle quality.

9. The method of claim 8, wherein the muscle quality is a fat fraction of the muscle.

10. The method of claim 1, wherein the machine learning system is a multi-class classifier and is trained to segment at least three muscles of a shoulder of the patient.

11. The method of claim 1, further comprising pre-processing the obtained image prior to inputting the image into the trained machine learning system.

12. The method of claim 11, wherein the pre-processing comprises performing a contrast limited adaptive histogram equalization (CLAHE).

13. The method of claim 1, wherein the skeletal muscles include a shoulder muscle.

14. The method of claim 1, wherein the skeletal muscles include a knee or thigh muscle.

15. The method of claim 1, wherein the skeletal muscles include an elbow muscle.

16. The method of claim 1, wherein determining the treatment procedure comprises determining a likelihood of success of a plurality of different treatment procedures and identifying the treatment procedure having the greatest likelihood of success.

17. The method of claim 1, wherein the image of skeletal muscles is a two-dimensional image from a computed tomography or magnetic resonance three-dimensional volume.

18. The method of claim 1, comprising inputting a plurality of images into the machine learning system, the plurality of images being of a three-dimensional volume of the skeletal muscles, wherein the machine learning system is trained to segment the muscles in the three-dimensional volume.

19. The method of claim 1, comprising inputting a plurality of images into the machine learning system, the plurality of images being of a three-dimensional volume of the skeletal muscles, wherein the machine learning system is trained to output a three-dimensional volume in which the plurality of skeletal muscles are segmented.

20. A method comprising:
obtaining a plurality of two-dimensional images of skeletal muscles of a patient, the plurality of two-dimensional images being of a three-dimensional volume obtained via computed tomography (CT) or magnetic resonance imaging (MRI);
inputting the plurality of images into a machine learning system trained to segment the muscles in the image and to output a three-dimensional volume of segmented skeletal muscles;
determining a fat fraction of at least one of the skeletal muscles based on an identification of the segmented muscles within the three-dimensional volume;
determining a likelihood of success of a plurality of different treatment procedures based on the determined fat fraction; and
identifying the treatment procedure having the greatest likelihood of success as a preferred treatment procedure for the patient.
